(19) United States
(12) Patent Application Publication
(10) Pub. No.: US 2020/0141951 A1
(43) Pub. Date: May 7, 2020

(54) VITRO DIAGNOSTIC METHOD FOR ALZHEIMER'S DISEASE BASED ON THE ALBUMIN REDOX LEVEL IN THE CEREBROSPINAL FLUID

(71) Applicant: GRIFOLS WORLDWIDE OPERATIONS LIMITED, Dublin (IE)

(72) Inventors: Salvador Grancha Gamon, Granollers (ES); Montserrat Costa Rierola, Mataro (ES); Ana Maria Ortiz Fernandez, Parets del Valles (ES)

(21) Appl. No.: 16/737,632

(22) Filed: Jan. 8, 2020

Related U.S. Application Data
(63) Continuation of application No. 15/804,689, filed on Nov. 6, 2017, now abandoned.

(30) Foreign Application Priority Data
Nov. 16, 2016 (ES) P 201631468

(51) Int. Cl. G01N 33/68

(52) U.S. Cl. CPC G01N 33/6896; G01N 33/6827; G01N 2800/2821; G01N 2333/765; G01N 33/6815; G01N 2333/76

(57) ABSTRACT

An in vitro method for diagnosing Alzheimer's disease (AD) includes determining the content of mercaptoalbumin (HMA) in a sample of cerebrospinal fluid (CSF), and comparing the content determined with the content of HMA in CSF in healthy subjects. If the HMA content is less than that of the healthy subjects, it is indicative of AD.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

AD is a progressive neurodegenerative disease, typically characterised by loss of short-term memory and other mental abilities (such as higher cognitive abilities) as the neurons die and various areas of the brain become atrophied. The disease tends to have an average duration of approximately 10 years from diagnosis, although said duration can vary in direct proportion to the severity of the disease at the time of diagnosis.

AD is the most common form of dementia; it is incurable and terminal, and occurs more frequently in persons aged over 65 years although it can also, in rare cases, develop as early as the 40s.

In general, the initial symptom is the inability to acquire new memories, but it tends to be confused with aging- or stress-related behaviour. When AD is suspected, the clinical diagnosis procedures available while the patient is living have up to now been based on guidelines and criteria established by the National Institute of Neurological Disorders and Stroke-Alzheimer's Disease and Related Disorders Association (NINCDS-ADRDA) (McKhann et al., 1984), mostly based on clinical opinion according to the results of neuropsychological tests, reports from the patient's family and friends and a general neurological assessment. The neurological examination can also include a study of brain images (neuroimaging).

### SUMMARY

The present invention relates to the sector of clinical diagnosis, and relates in particular to an in vitro method for diagnosing Alzheimer's disease (AD) based on determining the redox state of albumin in cerebrospinal fluid (CSF), in particular the content of mercaptoalbumin (HMA), which is the reduced form of albumin containing cysteine Cys-34 in the form of a free thiol group.

There is currently no definitive diagnostic method for AD, apart from post-mortem histological analysis of the patient's brain.

It is, however, known that human albumin is a non-glycosylated 66-kDa protein. Quantitatively, it is the most important protein in the blood plasma and its concentration in normal plasma is between 35 and 50 g/L, constituting up to 60% of total plasma proteins (Peters T. J.: All About Albumin; Biochemistry, Genetics, and Medical Applications. Academic Press, San Diego, 1996). Similarly, albumin is also the most abundant protein in the CSF, constituting around 67% of total proteins, with a concentration of around 200 mg/L (Edward J. Thompson, 2005, The roster of CSF proteins, PROTEINS OF THE CEREBROSPINAL FLUID: Analysis and Interpretation in the Diagnosis and Treatment of Neurological Disease, 2nd Ed. London, UK: Elsevier Academic Press.: 13-31).

The structure of human albumin consists of a polypeptide of 585 amino acids and approximately 67% alpha helices having no beta sheets (Otagiri M., Chuang V. T. Pharmaceutically important pre- and posttranslational modifications on human serum albumin. Biol Pharm Bull 2009; 32:527-534). Human albumin contains 6 methionine residues and 35 cysteine residues, the latter being involved in the formation of 17 disulfide bonds. Cys-34 is the only free cysteine in the whole molecule. Human albumin has specific antioxidant functions owing to its ability to bind to multiple ligands and its radical capture properties, both being closely related to its structure.

Although there are many possibilities for oxidising albumin, Cys-34 is a site that is particularly sensitive to oxidation/reduction. Therefore, it is generally legitimate to speak of the redox state of albumin in terms of Cys-34. Using chromatographic separation of albumin, three fractions are obtained depending on the redox state of the Cys-34 (Peters, 1996 cited above):
(i) the reduced form with cysteine in the form of a free thiol group, called human mercaptoalbumin (HMA);
(ii) the oxidised form with cysteine, forming a disulfide bond with a small compound containing a thiol group, mainly cysteine or cysteinyl-glycine, although it can also be with homocysteine and glutathione, called human non-mercaptoalbumin-1 (HNA1); and
(iii) the most oxidised form with cysteine as sulfinic or sulfonic acid, called human non-mercaptoalbumin-2 (HNA2).

In a healthy, sound person, approximately 61-69% of total albumin in plasma takes the form of HMA, 27-35% the form of HNA1 and 3-5% the form of HNA2 (Oettl K., et al. Oxidative damage of albumin in advanced liver disease. Biochim Biophys. Acta 2008; 1782: 469-473; Oettl K. and Marsche G. Redox State of Human Serum Albumin in Terms of Cysteine-34 in Health and Disease. Methods Enzymol. 2010; 474:181-95; and Oettl K. et al. Oxidative albumin damage in chronic liver failure: relation to albumin binding capacity; liver dysfunction and survival. J Hepatol, 2013, 59:978-983). It is generally believed that oxidisation of HMA and HNA1 is reversible, whereas oxidisation of HNA2 is an irreversible process.

Albumin can be subjected to various structural modifications, which means that its shape is modified and, therefore, its binding properties, as well as its redox state (Oettl, K. et al., 2010 cited above).

### BRIEF DESCRIPTION OF DRAWING

FIG. 1 shows amounts of HMA for a healthy person and a patient having AD.

### DETAILED DESCRIPTION OF EMBODIMENTS

The present invention is based on the surprising discovery that, in patients diagnosed with AD while they are living, the content of the reduced form of albumin (HMA) in the cerebrospinal fluid (CSF) is much lower than in healthy control subjects of an equivalent age range. This phenomenon has not been observed for the content of HMA in blood plasma, also when compared with healthy control subjects of an equivalent age range. Hence, it can also be said that it has been discovered that in patients diagnosed with AD while they are living, the difference or ratio between the HMA level in the CSF and in the plasma is increased compared with the same difference in healthy control subjects of an equivalent age range. Therefore, the two markers mentioned above, i.e. the content of HMA in CSF and the difference or ratio between the level of HMA in CSF and plasma, are useful for diagnosing AD.

As mentioned above, the definitive diagnostic test for AD has to be carried out post-mortem, and therefore diagnosis in a living patient is based on clinical opinion according to the results of neurological tests, reports from the patient's family and friends and a general neurological assessment. The test of the present invention therefore represents a diagnostic tool that is objective, quantifiable, easy to interpret, reliable, reproducible in different diagnostic centres, relatively inexpensive and not influenced by cultural aspects such as the patient's educational level, as is the case with some neuropsychological tests.

As used in the present document, “healthy subject” and its plural refer to subjects not suffering from AD.

Therefore, in a first aspect, the present invention relates to an in vitro method for diagnosing Alzheimer's disease (AD), comprising the following steps:
a) determining the content of mercaptoalbumin (HMA) in a sample of cerebrospinal fluid (CSF) from a patient; and
b) comparing the content determined in a) with the content of HMA in CSF in healthy subjects.

In a preferred embodiment, in step b) of the method of the present invention, if the HMA content determined in step a) is less than that of healthy subjects, it is indicative of AD; preferably the patient is diagnosed with AD.

In a preferred embodiment, both the patient and the healthy subjects mentioned above are human beings, preferably adult human beings.

In another preferred embodiment, the content of HMA in CSF is measured by high-performance liquid chromatography (HPLC) and fluorescence detection (FLD) using excitation and emission wavelengths of 280 and 340 nm respectively, based on the methodology described by Oettl K., 2010 (see above). HMA is quantified by taking account of the height of the relevant peak obtained in the corresponding chromatogram.

The cut-off value, determined by calculating the ROC curve after adjusting the sensitivity and specificity percentages to maximum values (91% and 100% respectively), below which it is considered that the HMA content measured in step a) is indicative of AD and/or leads to a diagnosis of AD in the patient, is preferably 37% (w/v).

In a second embodiment, the present invention relates to an in vitro method for diagnosing Alzheimer's disease (AD), comprising the following steps:
a) determining the content of mercaptoalbumin (HMA) in a sample of cerebrospinal fluid (CSF) and a sample of blood or plasma from a patient;
b) determining a difference or ratio between the content of HMA in the sample of CSF and the content of HMA in the sample of blood or plasma determined in step a); and
c) comparing the difference or ratio determined in step b) with the corresponding difference or ratio in healthy subjects.

In step b) of the method according to this second embodiment, it is envisaged that the difference determined is: the difference between the content of HMA in the sample of CSF and the content of HMA in the sample of blood or plasma determined in step a); or the difference between the content of HMA in the sample of blood or plasma and the content of HMA in the sample of CSF determined in step a). In a preferred embodiment, in step b) the difference between the content of HMA in the sample of CSF and the content of HMA in the sample of blood or plasma determined in step a) is determined in the form of a ratio (value of HMA in plasma/value of HMA in CSF).

When in step b) the difference is determined between the content of HMA in the sample of CSF and the content of HMA in the sample of blood or plasma determined in step a), in a preferred embodiment, in step c) of the method of the present invention, if the difference determined in step b), for example the ratio of the value of HMA in plasma / value of HMA in CSF (HMA plasma/HMA CSF), is greater than that of healthy subjects, it is indicative of AD; more preferably, the patient is diagnosed with AD. If the difference determined is something else, a person skilled in the art will make the necessary adaptations to the method of the present invention (for example, in relation to the comparison with the corresponding value of the difference in healthy subjects).

Additionally, and preferably, the sample of blood or plasma is a sample of plasma.

In a preferred embodiment, both the patient and the healthy subjects mentioned above are human beings, preferably adult human beings.

In another preferred embodiment, the content of HMA in CSF is measured by high-performance liquid chromatography (HPLC) and fluorescence detection (FLD) using excitation and emission wavelengths of 280 and 340 nm respectively, based on the methodology described by Oettl K., 2010 (see above). HMA is quantified by taking account of the height of the relevant peak obtained in the corresponding chromatogram.

The cut-off value, determined by calculating the ROC curve after adjusting the sensitivity and specificity percentages to maximum values (100%), from which it is considered that the difference determined in step b), for example the ratio of the value of HMA in plasma/value of HMA in CSF (HMA plasma/HMA CSF), is indicative of AD and/or that it leads to the diagnosis of AD in the patient, is preferably 1.1.

To aid understanding, the present invention is described in greater detail below, with reference to the attached figures, which are presented by way of example, and with reference to illustrative but non-limiting examples.

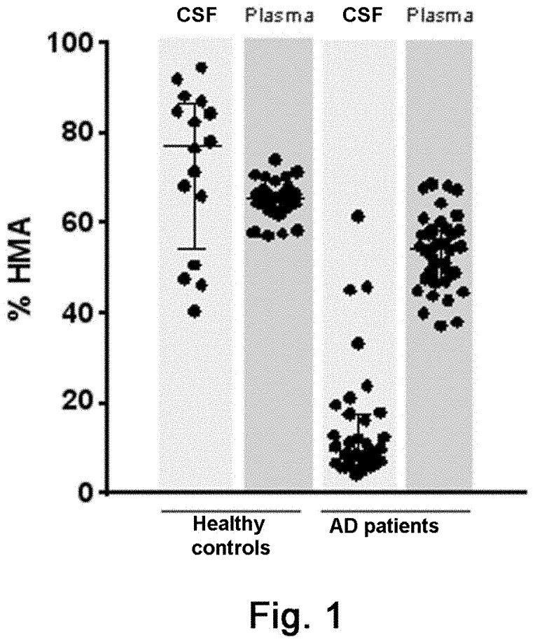

FIG. 1 shows the amount of HMA, by means of the peak height of HMA (as a percentage), obtained for samples of plasma and CSF from controls (healthy subjects, as defined above) and patients with AD. The ordinate axis (“y” axis) shows the peak height of HMA (as a percentage) and the abscissa axis (“x” axis) shows the group (the healthy controls on the left and the AD patients on the right). In addition, for each of the groups, the results obtained for the CSF samples are shown on the left and the results obtained for the plasma samples are shown on the right.

It is obvious to a person skilled in the art that if the content of HMA in CSF in AD patients is less than the content of HMA in CSF of healthy patients, the content of the two remaining fractions of albumin, according to the redox state of the Cys-34, i.e. of the HNA1 and/or the HNA2, will also be affected and can also serve as being indicative of AD.

### EXAMPLE

Example 1. Study of the diagnosis of Alzheimer's disease by measuring the content of HMA in CSF or the difference between said content and the content of HMA in plasma.

In the context of a multicentre, randomised, blind, controlled study in 42 patients who were being treated with therapeutic albumin and were diagnosed with mild-moderate AD, the present study was conducted to analyse the content of HMA in CSF and the difference between said content and the content of HMA in plasma.

For this, the content of HMA in baseline samples was measured (before the start of the clinical study) in the above-mentioned patients, both in CSF (N=34) and in plasma (N=37), as well as in the CSF (N=16) and plasma (N=37) of healthy subjects. The above-mentioned samples were in all cases obtained by lumbar puncture in the case of CSF and by drawing blood and then separating out the plasma in the case of the plasma samples, always following the standard medical procedures established for that purpose.

The samples of CSF and plasma mentioned were aliquoted and frozen at a temperature of −70° C. or below immediately after extraction. Before analysing the content of HMA, the samples were thawed at room temperature and, in the case of the plasma samples, the albumin concentration was determined by immunonephelometry or some other equivalent method. The HMA content was measured directly in the CSF samples whereas the plasma samples were diluted to a concentration of less than 10 mg/mL in phosphate buffer at pH 6.87. Next, the oxidised forms of the albumin were analysed by HPLC-FLD, as described above.

The results obtained are summarised in FIG. 1. As can be observed in said figure, the patients with AD show a great reduction in the content of HMA in CSF compared with the healthy subjects, whereas in plasma the reduction is slightly smaller. From the above, a deduction can be made of not only the diagnostic potential of the content of HMA in CSF but also the difference between said content and the content of HMA in plasma.

For AD patients, the median value obtained for the content of HMA in CSF is 9.6%, whereas the median of the content in plasma is 54.1%, the (HMA plasma/HMA CSF) ratio being 5.64. For healthy subjects, however, the median value of the content of HMA in CSF is 77.4% and that in plasma is 65.6%, the (HMA plasma/HMA CSF) ratio being 0.85.

What is claimed is:

1. An in vitro method for diagnosing Alzheimer's disease (AD), comprising the following steps:
a) determining the content of mercaptoalbumin (HMA) in a sample of cerebrospinal fluid (CSF); and
b) comparing the content determined in a) with the content of HMA in CSF in healthy subjects.

2. The method according to claim 1, wherein in step b), if the HMA content determined in step a) is less than that of the healthy subjects, it is indicative of AD.

3. The method according to claim 2, wherein in step b), if the HMA content determined in step a) is less than that of the healthy subjects, the patient is diagnosed with AD.

4. The method according to claim 1, wherein the cut-off value of HMA content, below which it is considered to be indicative of AD and/or leads to a diagnosis of AD, is 37%.

5. The method according to claim 1, wherein the sample comes from a human being.

6. The method according to claim 5, wherein said human being is an adult human being.

7. An in vitro method for diagnosing Alzheimer's disease (AD), comprising the following steps:
a) determining the content of mercaptoalbumin (HMA) in a sample of cerebrospinal fluid (CSF) and in a sample of blood or plasma from a patient;
b) determining a difference or ratio between the content of HMA in the sample of CSF and the content of HMA in the sample of blood or plasma determined in step a);
and
c) comparing the difference or ratio determined in step b) with the corresponding difference or ratio in healthy subjects.

8. The method according to claim 7, wherein in step b), the difference between the content of HMA in the sample of CSF and the content of HMA in the sample of blood or plasma determined in step a) is determined in the form of the ratio of the value of HMA in plasma/HMA in CSF (HMA plasma/HMA CSF).

9. The method according to claim 7, wherein in step c) of the method of the present invention, if the difference determined in step b) is greater than that of the healthy subjects, it is indicative of AD.

10. The method according to claim 9, wherein in step c) of the method of the present invention, if the difference determined in step b) is greater than that of the healthy subjects, the patient is diagnosed with AD.

11. The method according to claim 7, wherein, the cut-off value of the (HMA plasma/HMA CSF) ratio, above which it is considered to be indicative of AD and/or leads to a diagnosis of AD, is 1.1.

12. The method according to claim 7, wherein, the sample of blood or plasma is a sample of plasma.

13. The method according to claim 7, wherein, the sample comes from a human being.

14. The method according to claim 13, wherein said human being is an adult human being.
